(19) United States
(12) Patent Application Publication
(10) Pub. No.: US 2005/0010188 A1
(43) Pub. Date: Jan. 13, 2005

(54) Efficiently manufacturable absorbent disposable undergarment and method of manufacturing absorbent disposable article

(76) Inventors: Frank S. Glaug, Chester Springs, PA (US); Prelo M. Hood, Schwenksville, PA (US); Jocelyn P. Rice, Philadelphia, PA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 10/617,218

(22) Filed: Jul. 10, 2003

(51) Int. Cl. A61F 13/15

(52) U.S. Cl. 604/396

(57) ABSTRACT

A disposable absorbent article, e.g., a protective undergarment, and methods of manufacturing such articles. The undergarment is in the form of a chassis having front waist portion, a belly portion, a rear waist portion, a rear back portion, a crotch portion, and a pair of leg openings disposed on opposite sides of the crotch portion. Each of the leg openings has a high-cut concave edge portion at the front section, and a convex edge portion at the rear section. The concave edge portion of each leg opening is of complementary shape to the convex edge portion of that leg opening, so that the undergarment covers a substantial portion of the wearer's leg beneath the buttocks, while a substantial portion of the wearer's leg where the leg meets the torso is exposed.

### FIELD OF THE INVENTION

This invention relates generally to disposable absorbent undergarments, e.g., disposable underwear or briefs adults and youth and training pants for children, and more specifically to articles of that type that can be manufactured very efficiently and economically and which exhibit good protection from leakage, good fit and comfort.

### BACKGROUND OF THE INVENTION

Disposable absorbent articles, such as children's training pants, youth pants and adult incontinent briefs or underwear, are designed to absorb and contain body waste, e.g., urine and/or feces, to prevent such waste from soiling, wetting, or otherwise contaminating clothing or other articles, such as bedding, that come in contact with the wearer. Many such articles are commercially available. Moreover, the patent literature is replete with examples of various pant or brief constructions and methods of manufacturing them.

For example, In U.S. Pat. No. 4,764,234 (Smits, et al.), which is assigned to the same assignee as this invention and whose disclosure is incorporated by reference herein, there is disclosed a method of manufacturing an absorbent pad for utilization as a diaper or an adult incontinent brief. The method entails providing a moving web of material forming sequential backing sheets for the article. Nozzles are utilized to apply a pair of continuous bands of adhesive to the back sheet of the pad, prior to its assembly. The nozzles eject the adhesive onto a moving web which makes up the backing sheet and are shifted sideways during their extrusion of adhesive from a first location to a second location. This action generates a pair of non-linear bands of adhesive on the backing sheet. The nozzles are again shifted from their second location to their original first location. The elastic band is not shifted when the nozzles are, thus effectuating securement of the elastic bands only on the portions of the backing sheets where the adhesive was applied at the first location. The sequentially located sheets are cut on opposite marginal side to form leg cut-out areas, whereupon only those portions of the elastic bands at the center of each of the leg cut-out areas contract to form a gather thereat.

U.S. Pat. No. 4,764,242 (Gressick et al.), which is also assigned to the same assignee as this invention and whose disclosure is also incorporated by reference herein, discloses a machine for manufacturing a diaper or brief like that disclosed in the aforementioned Smits et al. patent.

U.S. Pat. No. 4,795,451 (Buckley), which is also assigned to the same assignee as this invention and whose disclosure is also incorporated by reference herein, discloses a disposable absorbent pad for use as a baby diaper or an adult incontinent brief wherein the backing sheet of the diaper has a pair of continuous bands of adhesive arranged in a stepped generally longitudinal configuration thereon. An elastic band is disposed over the mid-portion of the adhesive being stepped to the side, out of contact with the elastic. When the pads are cut in the assembly process, only that portion of elastic in contact with the adhesive causes the pad to gather. The remaining end portions of the adhesive bands are secured to the intermediate layer of absorbent fluff material, helping it remain in place, and act as a fluid barrier on the sides of the absorbent pad.

U.S. Pat. No. 4,804,379 (Toth et al.), which is also assigned to the same assignee as this invention and whose disclosure is also incorporated by reference herein, discloses a disposable absorbent pad for utilization as a diaper or adult brief. The pad is an absorbent member covered on one side by a sheet of fluid impervious material, and on the other side by a pair of generally parallel strips of fluid impervious material overlapping the absorbent pad and the backing sheet, in the crotch area to prevent leakage of fluid from the absorbent pad in the crotch area. The pad includes a pair of opposed leg cut-outs each of which is elasticized by a linear elastic band.

In U.S. Pat. No. 5,649,919 (Roessler et al.), there is disclosed an absorbent article which has a lateral width, a longitudinal length, longitudinally extending side margins, a front waistband portion, a back waistband portion, and an intermediate portion which interconnects the front and back waistband portions. The article comprises a backsheet layer and an absorbent retention portion superposed on the backsheet layer. A liquid permeable topsheet layer is superposed on the retention portion to sandwich the retention portion between the topsheet layer and the backsheet layer. An elasticizing means form elasticized gathers at leg opening portions of the article. The elasticizing means include a front set of laterally opposed, longitudinally extending leg elastic members located in the article side margins in at least the intermediate portion of the article. The front elastic members are arranged asymmetrically with respect to the article length and have a selected offset toward the front waistband portion of the article. A back set of laterally opposed, longitudinally extending leg elastic members are constructed separate from the front set of elastic members and are located in the article side margins in at least the intermediate portion of the article. The back elastic members are arranged asymmetrically with respect to the article length and have a selected offset toward the back waistband portion of the article.

In U.S. Pat. No. 5,904,675 (Laux et al.), there is disclosed an absorbent article has a longitudinal length dimension, a lateral cross dimension, a front waistband portion, a back waistband portion and an intermediate portion which interconnects the front and back waistband portions. The article includes a backsheet layer having a pair of laterally opposed side margins, with each side margin having an outwardly concave, terminal side edge contour located at appointed leg opening regions in an intermediate portion of each of the side margins. Each concave side edge contour has a selected longitudinal extent along the length dimension of the article. A liquid permeable topsheet layer is connected in superposed relation to the backsheet layer, and an absorbent body is sandwiched between the topsheet layer and the backsheet layer. A separate, elasticized and gathered leg gusset is connected to the article along each of the appointed leg opening regions, and each leg gusset is configured to extend beyond and to bridge between opposed, spaced-apart portions of an associated one of the concave side edge contours of the backsheet layer.

In U.S. Pat. No. 5,906,603 (Roe et al.), there is disclosed a disposable absorbent article including a topsheet, a backsheet, and an absorbent core having a pair of opposed longitudinal edges. The absorbent article includes a leg cuff extending at least laterally outwardly from each opposed longitudinal edge. Each leg cuff has a proximal edge, a distal edge and an elasticized region disposed between the proximal edge and the distal edge. The elasticized region has an inner edge, an outer edge and a width. The proximal edge of each leg cuff extends generally longitudinally along and adjacent to at least a portion of one of the opposed longitudinal edges and the distal edge of each of said leg cuffs is disposed laterally outwardly from the proximal edge. Each leg cuff includes an inner bond disposed adjacent at least a portion of the proximal edge of each leg cuff. An outer bond is spaced laterally outwardly from the inner bond, the spacing between the inner bond and said outer bond defining a leg cuff base width. Each leg cuff also includes an inner wall that extends upwardly and laterally outwardly from the inner bond to the inner edge of the elasticized region having a first height, and an outer wall that extends upwardly and laterally inwardly from the outer bond to the outer edge of the elasticized region having a second height. An elastic element is disposed in the elasticized region of each leg cuff and is provided generally parallel to the wearer's skin.

In U.S. Pat. No. 6,010,586 (Suprise), there is disclosed a disposable absorbent article which defines a first side portion, a second side portion, and a longitudinal centerline between the side portions. The absorbent article comprises an outer cover and an absorbent insert which is connected to the outer cover. The outer cover comprises a first side panel which is located in the first side portion of the absorbent article and a second side panel which is located in the second side portion of the absorbent article. An edge of the first side panel is connected to an edge of the second side panel to provide a seam which extends along the longitudinal centerline between the side portions of the absorbent article. The opposite waist regions on each side panel are configured to encircle the legs of the wearer and releasably engage together about the hips of the wearer., The disposable absorbent article has an aesthetically pleasing garment-like appearance and is readily refastenable about the hips of the wearer.

In U.S. Pat. No. 6,217,563 (Van Gompel et al.) there is disclosed an integral absorbent article, such as diaper. The diaper has a longitudinal article length and a lateral article width and includes an absorbent composite having first and second longitudinally opposed end regions, and at least a first longitudinally terminal end edge. The absorbent composite includes a substantially liquid-impermeable backsheet layer, a substantially liquid permeable topsheet layer, and a retention portion sandwiched between the backsheet and topsheet layers. The retention portion has laterally opposed, terminal side edges, and the absorbent composite includes a pair of laterally opposed side margins which extend laterally beyond the side edges of the retention portion. A first body panel is joined to the first longitudinal end of the absorbent composite. The first body panel has a body side surface, an outward surface, a panel length which is less than the article length, an outboard terminal end edge, and a relatively inboard terminal end edge. Each of the side margins of the absorbent composite is inwardly turned to provide a turned bodyside surface and a turned outward side surface of each side margin. At least a first portion of the turned bodyside surface of each side margin of the absorbent composite is secured to the outward surface of the first body panel with a body panel attachment.

In International Patent Application WO 00/37010 (Roessler et al.) there is disclosed a pant-like, refastenable, disposable absorbent article that includes an absorbent chassis and a pair of opposed side panels. The absorbent chassis defines a pair of laterally opposed side edges (30) and a pair of longitudinally opposed waist edges. The side panels extend between the side edges of the absorbent chassis to define a waist opening and a pair of leg openings in the pant-like disposable absorbent article. Each of the opposed side panels defines a first side margin which is permanently attached to the side edge (30) of the absorbent chassis in a back waist region of the absorbent article to provide a permanent joint. Each of the opposed side panels further defines a second side margin opposite the first side margin which is refastenably attached to the side edge of the absorbent chassis in the front waist region of the absorbent article to provide a refastenable joint. This application also discloses the process of making the article. In particular, the process entails providing a continuous web of interconnected absorbent chasses. A pair of laterally opposed back panels are attached to the side edges of each of the absorbent chasses in one of the front waist region or the back waist region to provide a permanent joint. A pair of laterally opposed front panels are refastenably attached to the side edges of each of the absorbent chasses in the opposite waist region of the absorbent article to provide a refastenable joint. The continuous web of interconnected absorbent chasses are cut into discrete absorbent articles and are folded about a fold line extending in a lateral direction through the crotch region of the article, thereby positioning the front panels and back panels in a facing relationship. The front and back panels are fastened together along a pair of laterally opposed side seams to define a waist opening and a pair of leg openings for the pant-like, refastenable, disposable absorbent article.

In U.S. Pat. No. 6,478,786 (Glaug), which is assigned to the same assignee as this invention, there is disclosed an absorbent article, e.g., a diaper, having a crotch portion, a waist portion, a front belly portion and a rear back portion. The waist, belly and back portions include plural transversely oriented elastic threads. The diaper includes an absorbent core. Longitudinally oriented elastic threads are located on opposite sides of the core and intersect the transverse elastic threads to enclose the core. The diaper includes a pair of leg openings, each of which has a first arcuate section, a second arcuate section, and an intermediate section. The first arcuate section includes at least one arcuate elastic thread, as does the second arcuate section. Those threads are spaced by a gap. A portion of the longitudinally oriented elastic threads on either side of the core is located closely adjacent a respective gap between the arcuate elastic threads to elasticize the leg openings.

In U.S. Pat. No. 6,514,233 (Glaug et al.), which is assigned to the same assignee as this invention, there is disclosed a disposable absorbent article, e.g., a diaper, and a method of manufacturing plural diapers. Each diaper includes a front section, a rear section and an intermediate section. Each section is a generally planar member formed of a flexible material having a top edge, a bottom edge and an opposed pair of side edges. The bottom edge of the front section is somewhat concave, and the bottom edge of the rear section is somewhat convex. The intermediate section is formed of a fluid pervious cover-stock, a fluid impervious barrier layer, and an absorbent core interposed therebetween. The intermediate section is of generally rectangular shape having a pair of elasticized sides and an pair of end portion one of which is adhesively secured to the middle of the front section, while the other end portion is similarly secured to the middle of the rear section. The front and rear sections are arranged to be releasably secured to each other by fastening tapes to mount the diaper. A portion of the bottom edge of the front section, one side of the intermediate section and a corresponding portion of the bottom edge of the rear section form one leg hole of the diaper. Corresponding portions of the front, rear and intermediate sections form the other leg hole. The tensioned intermediate section forms a pair of upstanding walls conforming to the crotch of the wearer to prevent leakage from the diaper. The diapers are made by providing plural sheet units, each making up a front section of one diaper and a rear section of the next succeeding diaper. Those units are adhesively secured to respective ends of an intermediate section in a sequential line of such sections. Then the two sheet units that are secured to the intermediate section are each severed transversely to complete a diaper. This process is repeated for succeeding diapers.

While the prior art disposable absorbent diapers and briefs may be generally suitable for their intended purposes, they still leave much to be desired from the standpoints simplicity of construction and of ease and economy of manufacture. Thus, a need presently exists for a child's training pants, a youth's pants and an adult brief or underwear that is simple in construction, can be manufactured economically and efficiently without wastage, and which provides a good fit, resistance to leakage and wearing comfort. It is to those ends that this invention is directed.

### SUMMARY OF THE INVENTION

One aspect of this invention is directed to disposable absorbent undergarment, e.g., a child's training pants, a youth's pants or an adult brief, arranged to be worn by a wearer to trap and collect waste products. Another aspect of this invention is directed to a method of economically manufacturing a disposable absorbent article, such as a diaper or an undergarment.

The undergarment basically comprises a pants-shaped chassis having a front waist portion, a belly portion, a rear waist portion, a rear back portion, a crotch portion, and a pair of leg openings disposed on opposite sides of the crotch portion. The crotch portion is located between the leg openings and has a front section and a rear section. The front waist portion and the rear waist portion are joined together at the sides of the chassis. The belly portion and the rear back portion are also joined together at the sides of the chassis.

Each of the leg openings has a high-cut concave edge portion at the front section, and a convex edge portion at the rear section. The concave edge portion of one of the leg openings is of complementary shape to the convex edge portion of the one of the leg openings. The concave edge portion of the other of the leg openings is of complementary shape to the convex edge portion of the other of the leg openings.

With this construction when the undergarment is worn it covers a substantial portion of the wearers leg beneath the buttocks, while a substantial portion of the wearer's leg where the leg meets the torso is exposed.

The method aspect of this invention entails manufacturing plural disposable absorbent articles, e.g., undergarments, like those described above, or diapers, in an economical manner. The method basically comprises the steps of providing a web of material having a first longitudinal axis, a pair of generally linear side edges, a first elongated elastic member extending generally linearly parallel to the first longitudinal axis adjacent one of the side edges, a second elongated elastic member extending generally linearly parallel to the first longitudinal axis adjacent the other of the side edges, at least a third undulating curved elastic member extending generally parallel to the first longitudinal axis adjacent the first elastic member, and at least a fourth undulating curved elastic member extending generally parallel to the first longitudinal axis adjacent the second elastic member. The web is severed along a longitudinal line between the third and fourth elastic members and generally parallel to the first longitudinal axis to form a front web section and a rear web section. The front and rear web sections are separated from each other. An intermediate section is provided having a second longitudinal axis, a pair of marginal side edges extending generally parallel to the second longitudinal axis, a front edge portion, a rear edge portion, a first elongated elastic member extending generally parallel to one of the marginal side edges, and a second elongated elastic member extending generally parallel to the other of the marginal side edges. The front edge portion of the intermediate section is secured to the front web section and the rear edge portion of the intermediate section is secured to the rear web sections so that the second longitudinal axis is generally perpendicular to the first longitudinal axis. The front and rear web sections are severed along respective transverse lines extending perpendicular to the first longitudinal axis on opposite sides of the insert section.

If the article is an undergarment the method also entails folding the intermediate section to bring portions of the front and rear sections contiguous with the transverse lines into engagement with each other and securing those sections together, so that the severed front web section forms a front portion of the undergarment and the severed rear web section forms a rear portion of the undergarment.

### DESCRIPTION OF THE DRAWING

FIG. 1 is a front view of one exemplary embodiment of a protective undergarment constructed in accordance with this invention;

FIG. 2 is a rear view of the protective undergarment shown in FIG. 1;

FIG. 3 is an illustration of one initial step in the manufacturing process for making the protective undergarment shown in FIGS. 1 and 2, wherein were the two webs of material are adhesively secured with the elastic threads interposed between the webs;

FIG. 4 is an illustration showing the assembled web at a further step in the manufacturing process for making the protective undergarment shown in FIGS. 1 and 2, wherein the assembled web is are shown prior to being severed to form a front section web and a rear section web;

FIG. 5 is an illustration, like FIG. 4, but showing the line along which the web is severed to form the front and rear web sections;

FIG. 6 is an illustration, like FIGS. 4 and 5, but showing the web at a later point in time after it has been severed into the front and rear web sections;

FIG. 7 is an illustration, like FIGS. 4-6, but showing the web sections at a still later point in time wherein an intermediate section is registered thereon to form the crotch portion of the undergarment;

FIG. 8 is an illustration, like FIGS. 4-7, but showing the web sections at a still later point in time wherein the assembled item has been folded at its intermediate section so that the front web section and the rear web sections overlie each other;

FIG. 9 is an illustration, like FIGS. 4-8, but showing the web sections at a still later point in time wherein the folded assembly has been sealed at respective sides of one of the items the undergarment, with the upstream most item of undergarment shown being severed from the line of the remaining items to complete one item of undergarment; and

FIG. 10 is an enlarged sectional view taken along line 10-10 of FIG. 7

### DETAILED DESCRIPTION OF THE PREFERRED EMBODIMENT

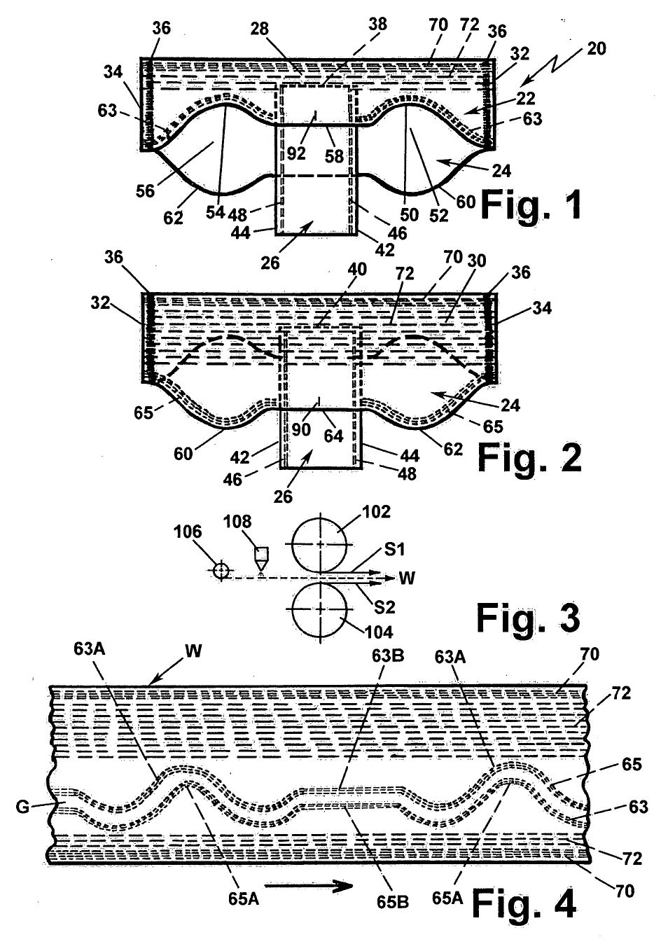

Referring now to the various figures of the drawing wherein like reference characters refer to like parts, there is shown in FIGS. 1 and 2 a disposable absorbent article 20 in the form of a protective undergarment, such as a child's training pants, a youth's pants or an adult brief, that is constructed in accordance with one exemplary embodiment of this invention. It should be pointed out that as used herein the term “undergarment” means an article that is in the form of a pants or brief to enable it to be readily pulled on or pulled off, like conventional underwear. Moreover, the term “disposable” means that the undergarment is designed to be used until soiled, either by urination or otherwise, and then discarded, rather than being washed and used again. The undergarment 20 is formed of the same basic materials as that used to form the undergarment of U.S. Pat. No. 6,478,786, whose disclosure is incorporated by reference herein.

To that end the undergarment 20 comprises a chassis including front portion or section 22, a back portion or section 24 (FIG. 2), and part of an intermediate section 26 that forms the crotch portion of the undergarment. As can be seen in FIG. 1, the front section 22 includes a waist and adjacent belly area 28 including plural transversely extending elastic threads (to be described later). The rear section 24 also includes a waist and adjacent lower back area 30 including plural transversely extending elastic threads 30 (also to be described later). The front section 22 and the rear section 24 are joined together at their respective side edges 32 and 34 by seals 36 extending along those side edges. The seals can be formed by any suitable means or technique, e.g., heat sealing, ultrasonic sealing, adhesive sealing, etc.

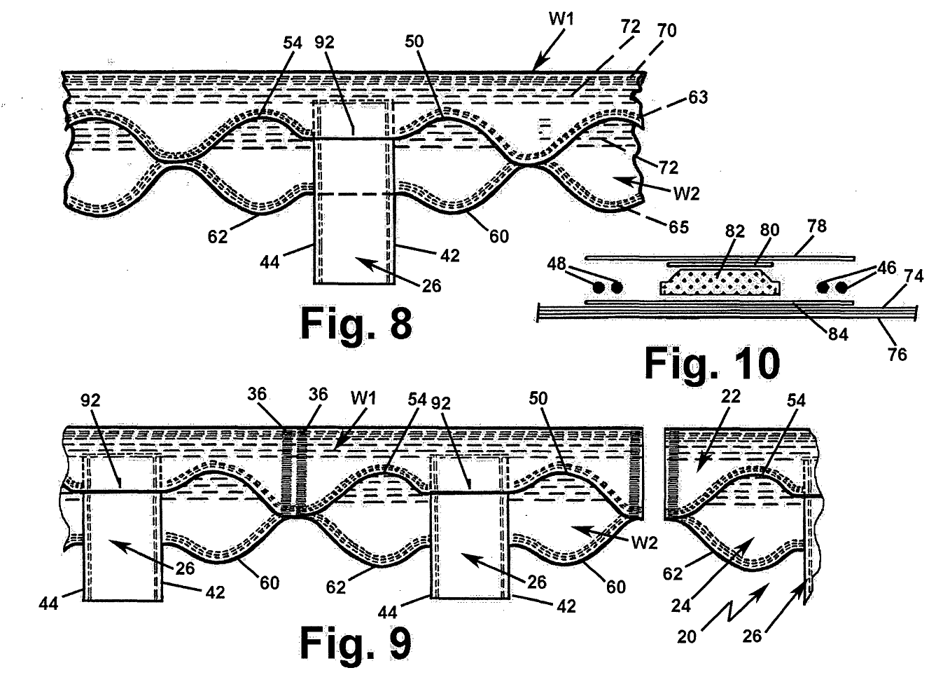

The intermediate section 26 will be described later with reference to FIG. 10. Suffice it for now to state that the section 26 is a generally planar, rectangularly shaped member having a front top edge 38 (FIG. 1) a back or rear top edge 40 (FIG. 2) and an opposed pair of linear side edges 42 and 44. A pair of elastic threads 46 are adhesively secured to the section 26 along its side marginal edge 42 and another pair of elastic threads 48 are adhesively secured to the section 26 along its side marginal edge 44. As will be described later, the threads 46 and 48 are pretensioned, i.e., secured to the section while under tension. The intermediate section 26 is adhesively secured to the inner surface of the front section 22 by an adhesive located in the area contiguous with the front top edge 38. In a similar manner, the intermediate section is adhesively secured to the inner surface of the back section 24 by an adhesive located in the area contiguous with the back top edge 40.

The lower edge of the front section 22 includes a portion which is somewhat S-shaped and is contiguous with the side 32. The S-shaped portion includes a concave recess 50. This recess forms a high-cut upper front portion of one of the undergarment's leg openings. That opening is designated by the reference number 52. In a similar manner a mirror image S-shaped arcuate portion is provided in the lower edge of the front section 22 contiguous with the side 34. This arcuate portion includes a concave recess 54 that forms a high-cut upper front portion of the other of the garment's leg openings 56. The remainder of the lower edge of the front section is a linear portion 58 located between the recesses 50 and 54.

The lower edge of the rear or back section 24 includes a portion which is somewhat S-shaped and is contiguous with the side 34. This arcuate portion includes a convex projection 60 forming a lower rear portion of the undergarment's leg opening 52. In a similar manner a mirror image S-shaped arcuate portion is provided in the lower edge of the back section 24 contiguous with the side 34. This arcuate portion includes a convex projection 62 forming a lower rear portion of the undergarment's leg opening 56. The remainder of the lower edge of the rear section 24 is a linear portion 64 located between the projections 60 and 62.

Plural pretensioned elastic fibers 63 are adhesively secured along the arcuate portions of the bottom edge of the front section 22 and plural pretensioned elastic fibers 65 are adhesively secured along the arcuate portions of the bottom edge of the rear section 24. These fibers extend parallel to each other and follow the shape of the adjacent lower edge of the garment's front or rear section at which they are located.

With the intermediate section 26 secured to the front and rear sections 22 and 24, respectively, the leg opening 52 is established by the concave recess 50 of the front section 22, the contiguous linear edge 42 of the intermediate section 26 and the convex projection 60 of the rear section 24. In a similar manner the other leg opening 56 of the undergarment is established by the concave recess 54 of the front section 22, the contiguous linear edge 44 of the intermediate section 26 and the lower projection 62 of the rear section 24. Each of these leg openings is elasticized by the elastic fibers located adjacent the various portions of each of those openings.

As will be appreciated by those skilled in the art, the elasticized leg openings 52 and 56 exhibit a high-cut front portion to ergonomically accommodate the portion of the wearer's leg that merges with his/her torso. This enables the wearer to comfortably sit or stand while wearing the garment, yet provides a sufficiently tight seal to preclude the egress of liquid out through the leg opening. The projections 62 and 64 of the leg openings serve to cover a substantial portion of the wearer's body where his/her buttocks merge with the upper leg, thereby providing very good coverage of the buttocks to collect and retain waste produced by the wearer.

As will be discussed later, the front and rear sections 22 and 24, respectively, are created at the same time from a single web (of either single ply or layer or multiple plies or layers) that is severed longitudinally along a single line, with the line forming the lower edge of the front section and the lower edge of the rear section. Thus, the two sections are of complementary shape.

As can be seen in FIG. 3, the front and rear sections of the chassis are composed of a pair of continuous webs or sheets S1 and S2 and are joined to produce a web W (to be described later). This web serves to form the “backsheet” of the undergarment. In this exemplary embodiment, the back sheet is a two-ply member formed of a cloth-like, non-woven, breathable material (to be described later).

As will be appreciated from the discussion to follow, the undergarment 20 is elasticized by the transversely extending elastics mentioned earlier to enable it to be readily pulled on and pulled off, and when in place to conforms to the body of the wearer, while also precluding the egress of any liquid such as urine or loose feces from it. To that end, the front and rear sections of the chassis are elasticized by the use of a plurality of elastic fibers or filaments which are glued in place between two webs of material S1 and S2 used to form a portion of the undergarments. In particular, as best seen in FIG. 2, a first group or plurality of elastic filaments or fibers 70 are included in the chassis and extend transversely across the chassis from side 32 to side 30 of the front section 22 immediately adjacent its top edge. The portion of the front section 22 contiguous with the top edge will be referred to as the front waist section. The portion of the front section below the front waist section will be referred to as the front belly section. The portion of the rear section 24 contiguous with the top edge will be referred to as the rear waist section and the portion of the rear section below it will be referred to as the rear back section. A similar group of elastic fibers 70 are included in a similar manner in the rear waist section (FIG. 2).

The transversely oriented elastic fibers 70 of both the front and rear waist sections are closely spaced parallel to one another, e.g., 0.25 inch. A second group or plurality of identical elastic fibers 72 are also included in the chassis and extend across the chassis from side 32 to side 34 in the front belly section and in the rear back section. The transversely oriented, second group of fibers 72 in each such group are also equidistantly spaced from one another, but at a greater spacing than the fibers 70 of the first groups, e.g., at 0.5 inch spacing.

As mentioned earlier, the fibers 63, 65, 70 and 72 are included in the chassis under tension (they are stretched) and as shown in FIG. 3 are placed between the inner surfaces of the two webs S1 and S2. Those webs are provided from rolls 102 and 104. The elastic threads making up the fibers 63, 65, 70 and 72 are provided from a roll or rolls 106 and are stretched. An applicator 108 provides a suitable adhesive to secure the fibers in place between the webs S1 and S2. The two webs with the interposed stretched fibers are adhesively secured to each other by any suitable adhesive to complete the two-ply web W of material from which the front and rear sections 22 and 24, respectively, are formed.

The material forming the front and rear section of the chassis can be any suitable material used in disposable absorbent articles. In the interest of wearing comfort, it is preferably a non-woven, cloth-like breathable material, such as spunbond/meltblown/spunbond polypropylene at 15 gsm available from Avgol of Holon, Israel. The material making up the chassis may be a polylaminate, e.g., polyfilm bonded to a non-woven material. The adhesive for securing the webs S1 and S2 together with the elastic fibers interposed therebetween, may be a construction adhesive, such as that available from National Starch and Chemical Company of Bridgewater, N.J.

Referring now to FIGS. 1, 2 and 10, the details of the construction of the intermediate section 26 will now be discussed. In particular, the section 26 comprises two plies or layers 74 and 76 of the same material as used to form the front and rear sections 22 and 24, respectively. These layers, along with the front and rear sections 22 and 24, respectively, make up the undergarment's chassis. The intermediate section 26 also includes a liquid pervious insert sheet or cover sheet 78, a fluid acquisition layer 80, a liquid absorbent core 82, and a liquid impervious barrier sheet or layer 84. The core 82 is located in a compartment 85 (to be described later) which is formed between the insert sheet 78 and the inner sheet 74 of the front and rear sections 22 and 24, respectively, as well as certain of the heretofore identified elastic fibers. In particular, as best seen in FIG. 7, the compartment 85 is defined by the intersection of the longitudinally oriented elastic fiber pairs 46 and 48 and the lowermost ones of the transversely oriented elastic fibers 72 of the front belly section and the higher most of ones of the transversely oriented elastic fibers 72 of the rear back section of the chassis. These intersecting fibers closely surround the core 82 to effectively form a gasket about the periphery of the core to prevent leakage of urine out of the core.

In order to prevent the egress of urine out through the non-woven sheets of the chassis, the intermediate section 26 also includes the heretofore identified barrier sheet or layer 84. The barrier sheet 84 is interposed between the core and the chassis and is formed of a liquid impervious material, e.g., a microporous polyfilm which is vapor permeable (breathable). In the embodiment shown herein, the barrier sheet is of the same generally rectangular shape and size as the cover or insert sheet 78 and is located in the crotch section of the undergarment within the compartment formed by the intersecting elastic threads. In accordance with one exemplary embodiment, the barrier sheet 84 is formed of a film of 0.5 mil polypropylene available from Pliant Corporation of Williamsburg, Va.

The core 82 is disposed on top of the barrier sheet 84. In the embodiment shown herein, the core 82 is of a generally rectangular shape. If desired, it may have a central portion of increased thickness as compared to its sides marginal portions. The thickened central portion of the core 82 may taper from the front to the rear. These structural features of the core and its location ensures that the maximum thickness of the core is located at the anatomical position at which the urine insult from the wearer will be initiated.

The core 82 can be formed of any suitable material(s), e.g., an airlaid composite, containing pulp, superabsorbent particulates and/or fibers, and binders. The binders may be chemical or thermal.

In accordance with one exemplary embodiment, the core 82 is made up of mainly cellulosic fibers, e.g., wood pulp fluff made of up bleached sulfate wood pulp containing soft wood fibers, such as that available from International Paper of Tuxedo, N.Y., co-mingled with hydrogel polymer particulates (known as Super Absorbent Polymer or “SAP”) such as cross-linked polyacrylate IM-3900 available from BASF Corporation of Charlotte, N.C. If desired, these materials may be optionally enwrapped in tissue. The amount of each absorbent material and SAP/fluff ratio depends on the size of the protective underwear, whether it is used for children (e.g., training pants) or for adults, and whether or not a transfer or fluid acquisition layer component is to be included in the section 26. In this regard, the fluid acquisition layer 80 as shown herein may be omitted from the undergarment 20, if desired. However, in the embodiment shown the acquisition layer 80 is used between the core 82 and the insert sheet or layer 78. The fluid acquisition layer is located over the thickened portion of the core, i.e., the portion where the urine insult will be initiated. As is known, the fluid acquisition layer 80 serves to manage, transport, accommodate and/or direct high volumes and flow rates of urine into the core.

The fluid acquisition layer 80 can be thru-air bonded/carded web, a spunbond bicomponent non-woven web, a web of crosslink cellulosic fibers, apertured 3D (three dimensional) film or the like. One particularly suitable material is a thru-air bonded bicomponent with a fast finish surfactant available from PGI Non-wovens of Landisville, N.J. and has an overall basis weight of 40 gsm. The bicomponent fibers are made of a polypropylene inner core and a polyethylene outer sheath. The fluid-acquisition layer 80 may be adhesively secured in place by any suitable construction adhesive, such as 34-373A adhesive available from National Starch and Chemical of Bridgewater, N.J. If desired, the core 82 may also be held in place by a similar adhesive.

In accordance with one exemplary embodiment of the undergarment 20, the insert sheet 78 is a non-woven spunbond polypropylene of 15 gsm available from Avgol of Holon, Israel, wherein the non-woven material is zone coated, with its longitudinal central area being hydrophillic and its two longitudinal side areas being hydrophobic. The insert sheet may also be formed of an apertured three dimensional film or a combination of such film and a non-woven material. The insert sheet 78 is of the same size and shape as the barrier sheet 84 and is disposed thereover. The insert sheet 78 is glued to the barrier sheet with the acquisition layer 80, the core 82 and the pairs of elastic fibers 46 and 48 interposed therebetween and completely about the perimeter of the core and acquisition layer. The transversely extending fibers 70 are formed of 620 Decitex LYCRA® and the transversely extending fibers 72 are formed of 800 Decitex LYCRA® available from E. I. DuPont de Nemours of Wilmington, Del. The longitudinally extending fibers 46 and 48 are formed of 800 Decitex LYCRA® and fibers 63 and 65 are formed of 940 Decitex LYCRA®.

The use of linearly disposed elastic fibers 46 and 48 in the intermediate section 26 and the arcuately disposed elastic fibers 63 and 65 in the front and rear sections 22 and 24, respectively, cooperate with one another to effectively form a complete peripheral seal to elasticize the leg openings 52 and 56 in lieu of completely encircling the leg openings with an arcuate elastic fiber. As should be appreciated from the discussion to follow, this feature expedites the manufacture of the protective undergarment 20 on a continuous basis. Moreover, by making use of the linearly extending longitudinal elastic fibers 46 and 48 to form a portion of the leg openings, one is able also to use those fibers as a portion of a gasketing system to intersect the transverse fibers 72 to form the heretofore identified sealed compartment 85 to prevent leakage of urine from the core.

It should be pointed out at this juncture that the materials as described heretofore to make up the undergarment 20 are merely exemplary of numerous materials that can be used for the various components. Thus, other conventional materials can be used for the chassis, the insert, its components, and the elastic threads or fibers.

In order to facilitate the correct orientation of the undergarment to put it on, since it is asymmetrical (the rear portion is longer/higher than the front portion), at least some of the elastic fibers of either the front or back of the undergarment may be distinctively colored to be readily discernable from the color of the chassis material to contrast with the color of the chassis, thereby enabling a person to readily determine what is the front and what is the rear of the undergarment so that it can be put on properly.

To put the undergarment on all that is required is for the user to orient the garment in the appropriate direction and to stretch the waist and contiguous upper portion of the chassis to enable his or her leg to be extended through the appropriate leg opening and then through the other leg opening. Then the user can pull the undergarment up so that its waist is located at the wearer's waist and its crotch is located over the wearer's crotch. Thus, the underwear can be put on as easily as any conventional undergarment. Removal of the undergarment is accomplished in a similar manner.

As should be appreciated by those skilled in the art from the foregoing the absorbent articles of this invention are simple in construction, effective and are comfortable to wear.

The undergarments 20 as described above can be made in various ways in accordance with the method(s) of this invention. In fact, one method of this invention can be used to manufacture a disposable diaper which is somewhat similar in construction than the undergarment 20 described above, but whose side marginal edges 32 and 34 of the front and rear sections, 22 and 24, respectively, are not secured together. In such an embodiment securement means, such as adhesive tapes, Velcro® fasteners or any other conventional fasteners found in conventional disposable diapers, can be used.

Referring now to FIGS. 3-9, the details of one exemplary method to make absorbent articles, such as the undergarment 20, will now be described. To that end, the two webs of material S1 and S2 are provided from a pair of rolls 102 and 104 and are brought into a confronting relationship with each other. The elastic threads that make up the elastic fibers 63, 65, 70 and 72 are fed from a roll or reel or plural rolls/reels 106 between the webs S1 and S2 and those webs are secured together by an adhesive supplied by applicator 108 to form the unitary web W from which the front and rear sections of the chassis will be formed. The elastic threads forming the waist elastics 70 are fed between the sheets S1 and S2 in a linear fashion so that they extend parallel to each other closely adjacent the side marginal edges of the resulting web W as shown in FIG. 4. The elastic threads forming the belly and back elastics 72 are fed in a similar manner. All of those fibers are fed under tension so that they remain under tension when secured in place.

The fibers forming the elastics for the bottom of the front and rear sections are fed from the roll(s) 106 in an alternating undulating-linear pattern. In particular, as shown in FIG. 4, the threads forming the elastics 63 of the front section's lower edge are provided so that there is an undulating portion 63A followed by a linear portion 63B followed by a mirror image undulating portion 63A and so on. In a similar manner, the threads forming the elastics 65 of the rear section's lower edge are provided so that there is an undulating portion 65A followed by a linear portion 65B followed by a mirror image undulating portion 65A and so on. The linear portions 63B and 65B of the threads 63 and 65 are not adhesively secured to the webs S1 and S2, but are under tension (for reasons to be described later). The two undulating/linear groups of elastic thread lines are disposed parallel to but spaced from each other by a gap G of corresponding shape.

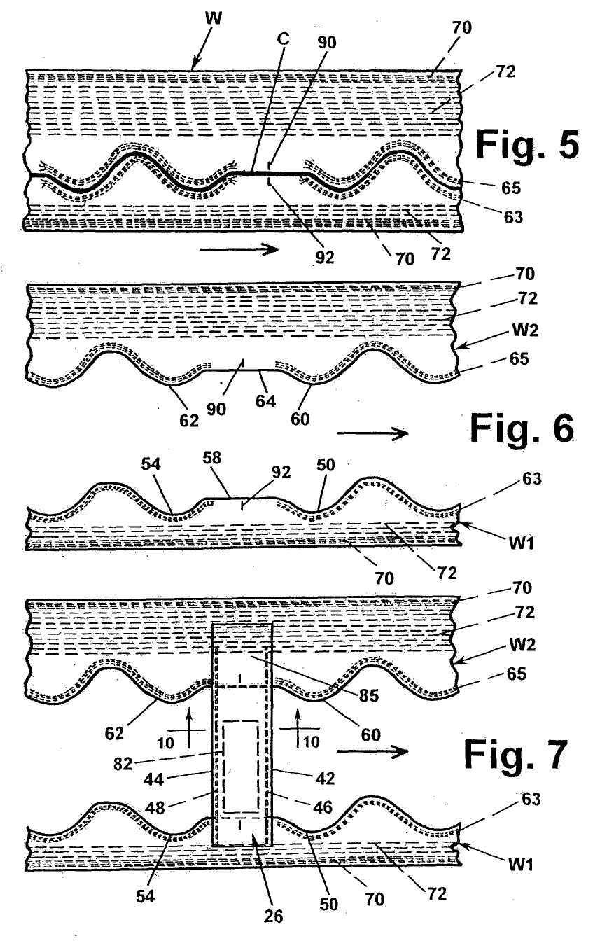

The web W shown in FIG. 4 is fed longitudinally in the direction of the arrow shown therein so that its leading end reaches a slitting station. The slitting station includes a knife or slitter (not shown) for severing the web W into two complementary shaped sections that will become the front section 22 and the rear section 24. To that end, the knife or slitter is moved with respect to the web W while the web is moving in the direction of the arrow so that the knife or slitter follows the path shown by the solid cut line designated by C in FIG. 5. As the web W is slit into the two sections another cutter reciprocates in and out to pierce through the web at 90 and 92 to form slits on opposite sides of the cut line C. This action severs the linearly extending portions 63B and 65B of the threads 63 and 65. Since, as mentioned earlier, those portions of the fibers 63 and 65 are not adhesively secured to the webs S1 and S2, but are under tension, the severed fibers 63 and 65 will snap back toward their contiguous undulating portions which are adhesively secured, leaving the linear portion between the undulating portions empty as shown in FIGS. 5 and 6.

The two sections of the web W are then separated from each other to form web sections W1 and W2 as shown in FIG. 6. These sections continue to move in unison in the direction of the arrow to a station for applying the intermediate section 26. At that station a section 26 is brought in and oriented so that its longitudinal axis is perpendicular to the longitudinal axis of the web sections W1 and W2. At this station the section 26 is disposed onto the inner surfaces 74 of the two web sections W1 and W2. An adhesive is applied at this station so that the portion of the section 26 contiguous with its end 38 is adhesively secured to the inner sheet 74 of the web section W1. At the same time and adhesive is applied so that the portion of the section 26 contiguous with its end 40 is adhesively secured to the inner sheet 74 of the web section W2.

Another method of securely attaching the intermediate section 26 onto the inner surfaces 74 of the two web sections W1 and W2 is to ultrasonically bond or heat seal the ends 38 and 40 of section 26 directly to the two web sections W1 and W2. The rest of the intermediate section 26, that is not ultrasonically bonded or heat sealed to web sections W1 and W2, yet in contact with them, can be secured with adhesive.

From this station the two web sections W1 and W2 and the adhesively secured intermediate section 26 move as an assembly in the direction of the arrow to a folding station. At the folding station the intermediate section 26 of the assembled unit is folded along a central line located between the outside marginal edges of the web sections W1 and W2 to bring those sections into a confronting relationship, as shown in FIG. 8.

From there the folded assembly moves to a final sealing and severing station. At that station the confronting web sections W1 and W2 are sealed together at two closely adjacent seal lines 36 on opposite, equidistantly spaced locations with respect to the intermediate section 26, i.e., at locations between the trailing arcuate edges 54 and 62 of the upstream assembly that will become an undergarment 20 and the leading arcuate edges 50 and 60 of the immediately downstream assembly. Once the heat or ultrasonic seals are formed, a severing knife or slitter (not shown) severs the sealed web sections W1 and W2 between the immediately adjacent seal lines 36, to thereby complete an undergarment 20. Each successive assembly is sealed and severed in the same manner to create successive undergarments, so that the undergarments 20 can be produced on a continuous basis.

It should be pointed out that while the method has been described for manufacturing undergarments on a continuous or mass-produced basis, it can be used to continuously mass produce diapers as well. In such an application the side edges of the assembled items, i.e., the items at the stage shown in FIG. 8, are not sealed together, but are left unconnected, and then are severed along their respective sides in a manner like described with respect to FIG. 9. The unsealed sides can then be provided with conventional diaper closures. In particular, fastening tapes or other conventional fasteners can then be applied to the respective sides of the back section of the diaper, to complete it. If the article made is a diaper the transversely extending elastic threads 70 and 72 may be omitted.

As should be appreciated by those skilled in the art, the undergarments 20 or diapers (as the case may be) can be manufactured in accordance with this invention with no wastage of material forming the various sections thereof. In particular, since the front section 22 and rear section 24 are of complementary shape they can be made at one time without any material wastage. Moreover, the elastics can be applied easily, without wastage. Thus, the subject method represents an extremely simple and economical manner of mass producing absorbent undergarments or diapers. Moreover, the undergarments produced exhibit outstanding ergonomic characteristics, without any sacrifice in absorbency and leakage protection. An additional advantage of the manufacturing process of this invention is its extreme flexibility to effect the efficient manufacture different types of absorbent articles without substantial modification to the manufacturing equipment. In particular, the construction of the front and rear sections can be altered as desired to create different undergarments or other similar absorbent articles. Different materials can be used to make the front and rear sections of the undergarment. If the absorbent article is a diaper, different types of fastening tapes (e.g., adhesive, multi-hook, etc.,) can be used, “landing zones” can be incorporated into the front section to facilitate the releasable securement of the fastening tapes thereto and to enable repeated fastening and re-fastening, if desired. Elastic materials can be provided in either or both of the front and rear sections along their top edges in the interests of forming a more secure or closer fit. The shape of the front and rear sections can be readily altered, if desired, by merely altering the pattern of the elastic threads elasticizing the lower edges of the front and rear sections and by correspondingly alternating the shape of the cut line C (although as discussed earlier it is preferable that the lower edge portion of the front panel be concave to result in a high leg opening in the interest of comfort and the lower edge portion of the rear panel be convex to cover the buttocks). So, too, the construction of the intermediate sections can be altered as desired, e.g., different materials can be used for the cover-stock and barrier walls, the core can be of different materials and shapes (e.g., some hour-glass shaped instead of rectangular), a fluid acquisition layer of any particular material or construction may be incorporated into the intermediate section between the cover-stock and the core, etc., standing leg cuffs can be added, etc.

Further economies can be achieved in the manufacturing process by making use of only a single layer or ply of material forming the chassis. Further still, the material making up the barrier wall in the crotch area, if a film, can be of lower gauge in the interest of economics, without the loss of functionality.

Without further elaboration the foregoing will so fully illustrate our invention that others may, by applying current or future knowledge, adopt the same for use under various conditions of service.

1. A disposable protective undergarment arranged to be worn by a person having legs to trap and collect loose or liquid waste products of the person, said undergarment comprising a pants-shaped chassis having a front waist portion, a belly portion, a rear waist portion, a rear back portion, a crotch portion, and a pair of leg openings disposed on opposite sides of said crotch portion, with said crotch portion being located between said leg openings and having a front section and a rear section, said front waist portion and said rear waist portion being joined together at the sides of said chassis, said belly portion and said rear back portion being joined together at the sides of said chassis, each of said leg openings having a high-cut concave edge portion at said front section, a convex edge portion at said rear section and an intermediate edge portion along said crotch portion, said concave edge portion of one of said leg openings being of complementary shape to said convex edge portion of said one of said leg openings, said concave edge portion of the other of said leg openings being of complementary shape to said convex edge portion of said other of said leg openings, whereupon when said undergarment is worn said undergarment covers a substantial portion of the wearers leg beneath the buttocks, while a substantial portion of the wearer's leg where the leg meets the torso is exposed.

2. The disposable protective undergarment of claim 1 wherein each of said leg openings is elasticized.

3. The disposable protective undergarment of claim 2 wherein each of said leg openings is elasticized by at least one elastic thread extending along said concave edge portion, said convex edge portion and along said intermediate edge portion.

4. The disposable protective undergarment of claim 1 additionally comprising a plurality of transversely extending elastic threads in said front waist portion, said belly portion and said rear waist portion.

5. The disposable protective undergarment of claim 3 additionally comprising a plurality of transversely extending elastic threads in said front waist portion, said belly portion and said rear waist portion.

6. The disposable protective undergarment of claim 1 additionally comprising an absorbent core.

7. The disposable protective undergarment of claim 5 additionally comprising an absorbent core and an insert sheet, and wherein certain ones of said transversely extending elastic threads intersect with said elastic threads of said leg openings to form an enclosed, gasketed compartment for said liquid absorbent core to prevent the egress of liquid therefrom.

8. The protective undergarment of claim 7 wherein said transversely oriented elastic threads comprise two first groups and two second groups, a first one of said two first groups being located in said front waist portion and a second one of said first two groups being located in said rear waist portion, a first one of said two second groups being located in said belly portion and a second one of said two second groups being located in said rear back portion, and wherein said transversely oriented threads of said first groups are spaced closer to one another than said transversely oriented threads of said second groups.

9. The protective undergarment of claim 1 wherein said crotch portion is a separate member having a pair of sides and a pair of ends, one of said ends being secured to said belly portion and the other of said ends being secured to said rear waist portion.

10. The protective undergarment of claim 9 wherein each of said leg openings is elasticized by at least one elastic thread extending along said concave edge portion, by at least one elastic thread extending along said convex edge portion and by at least one elastic thread extending along said an associated one of said pair of sides of said rectangularly shaped member.

11. The protective undergarment of claim 10 wherein said separate member forming said crotch portion is of generally rectangular shape.

12. The protective undergarment of claim 11 additionally comprising a plurality of transversely extending elastic threads in said front waist portion, said belly portion and said rear waist portion.

13. The protective undergarment of claim 10 additionally comprising a plurality of transversely extending elastic threads in said front waist portion, said belly portion and said rear waist portion.

14. The protective undergarment of claim 1 wherein said chassis comprises a non-woven material.

15. The protective undergarment of claim 14 wherein said non-woven material comprises spunbond polypropylene.

16. The protective undergarment of claim 1 additionally comprising a core formed of a highly moisture absorbent material.

17. The protective undergarment of claim 16 additionally comprising a fluid acquisition layer disposed over said core.

18. The protective undergarment of claim 14 additionally comprising a core formed of a highly moisture absorbent material.

19. The protective undergarment of claim 18 additionally comprising a fluid acquisition layer disposed over said core.

20. A method for manufacturing plural disposable absorbent articles, each of said absorbent articles comprising a front section, a rear section and an intermediate section, said method comprising the steps of:
(A) providing a web of material having a first longitudinal axis, a pair of generally linear side edges, a first elongated elastic member extending generally linearly parallel to said first longitudinal axis adjacent one of said side edges, a second elongated elastic member extending generally linearly parallel to said first longitudinal axis adjacent the other of said side edges, at least a third undulating curved elastic member extending generally parallel to said first longitudinal axis adjacent said first elastic member, and at least a fourth undulating curved elastic member extending generally parallel to said first longitudinal axis adjacent said second elastic member;
(B) severing said web along a longitudinal line between said third and fourth elastic members and generally parallel to said first longitudinal axis to form a front web section and a rear web section;
(C) separating said front and rear web sections from each other;
(D) providing an intermediate section having a second longitudinal axis, a pair of marginal side edges extending generally parallel to said second longitudinal axis, a front edge portion, a rear edge portion, a first elongated elastic member extending generally parallel to one of said marginal side edges, and a second elongated elastic member extending generally parallel to the other of said marginal side edges;
(E) securing said front edge portion of said intermediate section to said front web section and securing said rear edge portion of said intermediate section to said rear web sections so that said second longitudinal axis is generally perpendicular to said first longitudinal axis;
(F) severing said front and rear web sections along respective transverse lines extending perpendicular to said first longitudinal axis on opposite sides of said insert section.

21. The method of claim 20 wherein each of said absorbent articles is in the form of protective undergarment, wherein said method additionally comprises the steps of:
(G) folding said intermediate section to bring portions of said front and rear sections contiguous with said transverse lines into engagement with each other and securing said sections together thereat, so that said severed front web section forms a front portion of said undergarment or pants and said severed rear web section forms a rear portion of said undergarment.

22. The method of claim 21 wherein said method additionally comprises:
(G) providing a pair of web layers, each formed of a flexible material; and
(H) interposing said elastic members between said web layers and laminating said layers together to form said web of material.

23. The method of claim 21 wherein said elongated elastic members are provided in the form of elastic threads.

24. The method of claim 23 wherein said first elongated elastic member is one of a first group of plural elastic threads extending continuously generally linearly parallel to said first longitudinal axis adjacent one of said side edges, said second elongated elastic member is one of a second group of plural elastic threads extending continuously generally linearly parallel to said first longitudinal axis adjacent the of said side edges.

25. The method of claim 23 wherein said third elastic member is one of a third group of plural elastic threads extending generally parallel to said first longitudinal axis, and said fourth elastic member is one of a fourth group of plural elastic threads extending generally parallel to said first longitudinal axis.

26. The method of claim 25 wherein said third group of elastic threads are disposed in discontinuous segments spaced from each other in a direction parallel to said first longitudinal axis, and wherein said fourth group of elastic threads are disposed in discontinuous segments spaced from each other in a direction parallel to said first longitudinal axis.

27. The method of claim 26 wherein said method additionally comprises:
(G) providing each of said threads of said third group of elastic threads as a continuous member having undulating portions and linear portions interposed with each other and wherein each of said continuous members is under tension, with said undulating portions of each of said continuous threads being adhesively secured to said web of material and said linear portion of each of said continuous threads being unsecured to said web;
(H) providing each of said threads of said fourth group of elastic threads as a continuous member having undulating portions and linear portions interposed with each other and wherein each of said continuous members is under tension, with said undulating portions of each of said continuous threads being adhesively secured to said web of material and said linear portion of each of said continuous threads being unsecured to said web; and
(H) severing each of said threads of said third and fourth groups of elastic threads at said linear portions to cause said each of said threads of said third and fourth groups of elastic threads to be disposed in discontinuous segments spaced from each other in a direction parallel to said first longitudinal axis.

28. The method of claim 27 wherein said first elongated elastic member is one of a first group of plural elastic threads extending continuously generally linearly parallel to said first longitudinal axis adjacent one of said side edges, said second elongated elastic member is one of a second group of plural elastic threads extending continuously generally linearly parallel to said first longitudinal axis adjacent the of said side edges.

29. The method of claim 32 wherein said web of material is moved in a direction parallel to said first longitudinal axis.

30. The method of claim 29 wherein said web is severed along said longitudinal line between said third and fourth elastic members as it is moving.

31. The method of claim 30 wherein said intermediate sections are provided at spaced apart intervals on said front and rear web sections as said front and rear web sections are moving in a direction parallel to said first longitudinal axis.

32. The method of claim 31 wherein said intermediate section are secured said front and rear web sections as said web is moving.

33. The method of claim 32 wherein the folding of said intermediate section, the securing of said front and rear sections together and the severing of said sections thereat is accomplished as said web is moving.
